(19) United States
(12) Patent Application Publication
(10) Pub. No.: US 2010/0240070 A1
(43) Pub. Date: Sep. 23, 2010

(54) Nonseparation Assay Methods Using Peroxide Generating Enzymes

(75) Inventors: Hashem Akhavan-Tafti, Howell, MI (US); Michael Salvati, Minnetrista, MN (US); Kenneth P. Kapsner, Minneapolis, MN (US)

Correspondence Address: Michael C. Schiffer - Legal Dept.;BECKMAN COULTER, INC., 250 S. Kraemer Boulevard, BREA, CA, 92821, US

(73) Assignee: BECKMAN COULTER, INC., Brea, CA (US)

(21) Appl. No.: 12/741,619

(22) PCT Filed: Nov. 7, 2008
(86) PCT No.: PCT/US08/82790

§ 371 Date: May 6, 2010

Related U.S. Application Data
(60) Provisional application No. 60/986,191, filed on Nov. 7, 2007.

(51) Int. Cl. G01N 33/53; C12Q 1/28; C12Q 1/30

(52) U.S. Cl. 435/7.9; 435/28; 435/27

(57) ABSTRACT

Nonseparation assay methods using peroxide generating enzymes in combination with a solid support for analyte detection are disclosed. The present assay methods provide a high degree of sensitivity, are simple and efficient to perform, and are excellent tools for diagnostic and high through-put screening applications.

### CROSS-REFERENCES TO RELATED APPLICATIONS

This application claims the benefit of U.S. Provisional Application No. 60/986,191, filed on Nov. 7, 2007, the contents of which are herein incorporated by reference in their entirety for all purposes.

### STATEMENT AS TO RIGHTS TO INVENTIONS MADE UNDER FEDERALLY SPONSORED RESEARCH AND DEVELOPMENT

NOT APPLICABLE

### BACKGROUND OF THE INVENTION

Homogeneous assay formats avoid the need for separation of an added detectably labeled specific binding partner is used. This type of methodology relies on devising a detection principle that is either turned on or turned off as a result of the binding reaction. In contrast, heterogeneous assays formats rely on physical separation of bound and free detectably labeled specific binding partners before quantitation.

Homogeneous enzyme immunoassays generally exploit the antibody:antigen binding reaction to either activate or inhibit a label enzyme and may involve various methods of quenching fluorescence through antibodies or other fluorescent quenchers. Despite the considerable efforts made in devising homogeneous, or non-separation, assay formats, they still do not experience widespread commercial adoption. Heterogeneous assays are viewed as simpler to develop and mass-produce, even though they are operationally more complex. In particular, the field of high volume clinical immunodiagnostics and the smaller field of clinical nucleic acid diagnostics are dominated by heterogeneous assay formats. Within this arena, test formats would be beneficial to the field that could simplify protocols, reduce complexity and improve compatibility with automation by removing unnecessary steps. The present invention addresses these and other needs in the art.

### BRIEF SUMMARY OF THE INVENTION

The present invention provides simple and efficient assay methods of detecting analytes. The assays presented herein may be performed for applications such as diagnostics and high through-put screening procedures.

In one aspect, a novel method of detecting an analyte in a sample is provided. The method includes contacting the analyte with a solid support conjugate and a first analyte binder conjugate. The first analyte binder conjugate is a peroxidase enzyme conjugated to a first analyte binder. The solid support conjugate is a solid support that is conjugated to a second analyte binder, a peroxide generating enzyme, and a chemiluminescent compound. The analyte is allowed to bind to the first analyte binder and the second analyte binder thereby forming a detectable solid support bound analyte complex. The detectable solid support bound analyte complex is contacted with a peroxide generating enzyme substrate thereby producing a peroxide. The peroxidase enzyme is allowed to react with the peroxide which results in the activation of the chemiluminescent compound and the production of a chemiluminescent signal. The analyte is detected by detecting the chemiluminescent signal.

In another aspect, a method of detecting an analyte in a sample is provided. The method includes contacting the analyte with a solid support conjugate and a first analyte binder conjugate. The first analyte binder conjugate is a peroxidase enzyme conjugated to a first analyte binder and the first analyte binder is being bound to a competition analyte. The solid support conjugate is a solid support conjugated to a second analyte binder, a peroxide generating enzyme, and a chemiluminescent compound. The analyte and the first analyte binder conjugate are allowed to competitively bind to the second analyte binder. The binding of the first analyte binder conjugate to the second analyte binder forms a detectable solid support bound analyte complex. The detectable solid support bound analyte complex is contacted with a peroxide generating enzyme substrate thereby producing a peroxide. The peroxidase enzyme is allowed to react with the peroxide which results in the activation of the chemiluminescent compound and the production of a chemiluminescent signal. The analyte is detected by detecting the chemiluminescent signal.

In another aspect, a solid support conjugate is provided. The solid support conjugate includes a solid support conjugated to a chemiluminescent compound, a hydrogen peroxide generating enzyme, and an analyte binder.

In another aspect, a kit for detecting an analyte in a sample is provided. The kit includes a first analyte binder conjugate that is conjugated to a peroxidase enzyme, and a solid support conjugate that is conjugated to a second analyte binder, a peroxide generating enzyme, and a chemiluminescent compound.

### BRIEF DESCRIPTION OF THE DRAWINGS

FIG. 1 is a schematic representation of a chemiluminescent detection method including a solid support conjugate and a first analyte binder conjugate, a peroxide generating enzyme, a peroxide generating enzyme substrate, and a chemiluminescent compound.

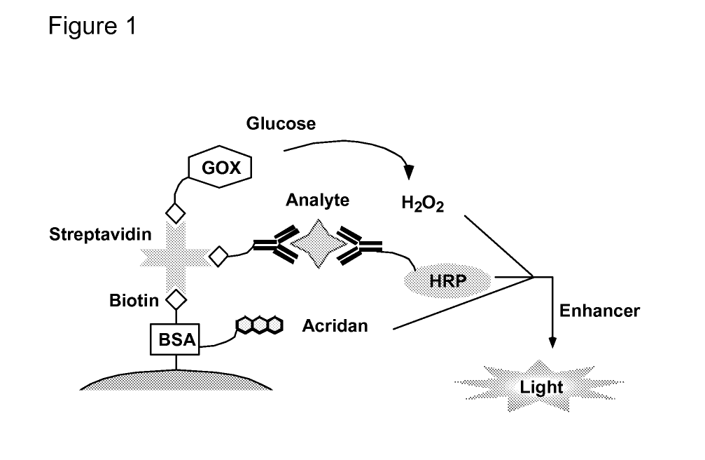

### DETAILED DESCRIPTION OF THE INVENTION

### I. Definitions

Generally, a “sample” represents a mixture containing or suspected of containing an analyte to be measured in an assay. Samples which can be typically used in the methods of the invention include bodily fluids such as blood, which can be anti-coagulated blood as is commonly found in collected blood specimens, plasma, urine, semen, saliva, cell cultures, tissue extracts and the like. Other types of samples include solvents, seawater, industrial water samples, food samples and environmental samples such as soil or water, plant materials, eukaryotes, bacteria, plasmids, viruses, fungi, and cells originated, from prokaryotes.

An “analyte” is a substance in a sample to be detected in an assay. The analyte can be a protein, a peptide, an antibody, or a hapten to which an antibody that binds it can be made. The analyte can be a nucleotide or oligonucleotide which is bound by a complementary nucleic acid or oligonucleotide. Other types of analytes include, drugs such as steroids, hormones, proteins, glycoproteins, mucoproteins, nucleoproteins, phosphoproteins, drugs of abuse, vitamins, antibacterials, antifungals, antivirals, purines, antineoplastic agents, amphetamines, azepine compounds, nucleotides, and prostaglandins, as well as metabolites of any of these drugs, pesticides and metabolites of pesticides, and receptors. Analytes also include cells, viruses, bacteria and fungi.

The term “specific binding” refers to binding between two molecules such as a ligand and a receptor and is characterized by the ability of a molecule (ligand) to associate with another specific molecule (receptor) in the presence of many other diverse molecules. Specific binding of a ligand to a receptor is also evidenced by reduced binding of a detectably labeled ligand to the receptor in the presence of excess of unlabeled ligand (i.e. a binding competition assay).

The term “antibody” (Ab) refers to a polypeptide with a framework region from an immunoglobulin gene or fragments thereof that specifically binds and recognizes an antigen. The recognized immunoglobulin genes include the kappa, lambda, alpha, gamma, delta, epsilon, and mu constant region genes, as well as the myriad immunoglobulin variable region genes. Immunoglobulin light chains are classified as either kappa or lambda, whereas immunoglobulin heavy chains are classified as gamma, mu, alpha, delta, or epsilon. The immunoglobulin heavy chains define the immunoglobulin classes (isotypes), IgG, IgM, IgA, IgD and IgE, respectively. Typically, the antigen-binding region of an antibody will be most critical in specificity and affinity of binding. Antibodies can be polyclonal or monoclonal, derived from serum, a hybridoma or recombinantly cloned, and can also be chimeric, primatized, or humanized.

An example of an immunoglobulin (antibody) structural unit includes a tetramer. Each tetramer is composed of two identical pairs of polypeptide chains, each pair having one “light” (about 25 kD) and one “heavy” chain (about 50-70 kD). Disulfide bonds connect the heavy chain and the light chain of each individual pair. Further, the two heavy chains of each binding pair are connected through a disulfide bond in the hinge region. Each heavy and light chain has two regions, a constant region and a variable region. The constant region of the heavy chain is identical in all antibodies of the same isotype, but differs in antibodies of different isotypes. The variable region located at the N-terminus of the heavy and the light chain includes about 100 to 110 or more amino acids and is primarily responsible for antigen recognition. The terms variable light chain (V_{L}) and variable heavy chain (V_{H}) refer to these light and heavy chains, respectively.

Antibodies exist, for example as intact immunoglobulins or as a number of well-characterized fragments produced by digestion with various peptidases. Thus, for example, pepsin digests an antibody below the disulfide linkages in the hinge region to produce F(ab)′₂, a dimer of Fab, which itself is a light chain joined to V_{H}—C_{H}1 by a disulfide bond. The F(ab)′₂ may be reduced under mild conditions to break the disulfide linkage in the hinge region, thereby converting the F(ab)′₂ dimer into a Fab monomer. The Fab monomer is essentially Fab with part of the hinge region (see Fundamental Immunology (Paul ed., 3d ed. 1993). While various antibody fragments are defined in terms of the digestion of an intact antibody, one of skill will appreciate that such fragments may be synthesized de novo either chemically or by using recombinant DNA methodology. Thus, the term antibody, as used herein, also includes antibody fragments either produced by the modification of whole antibodies, or those synthesized de novo using recombinant DNA methodologies (e.g., single chain Fv) or those identified using phage display libraries (see, e.g., McCafferty et al., Nature 348:552-554 (1990)).

A “chemiluminescent compound” as used herein, refers to a monovalent chemiluminescent compound conjugated to a solid support, comprising a chemiluminescent moiety and a linking moiety. The terms “chemiluminescent group” and “chemiluminescent moiety” are used interchangeably as are the terms “linking moiety” and “linking group.” The chemiluminescent moiety may undergo a reaction with an activator resulting in the conversion of the chemiluminescent moiety into a higher or excited state of energy. Without being bound by any particular mechanistic theory, the excited state may directly emit light upon relaxation or may transfer excitation energy to an emissive energy acceptor, thereby returning to the ground state. After being excited the emissive energy acceptor may emit light. A class of compounds which by incorporation of a linking moiety could serve as a chemiluminescent compound include, but is not limited to, cyclic diacylhydrazides such as luminol and structurally related cyclic hydrazides including isoluminol, aminobutylethylisoluminol (ABET), aminohexylethylisoluminol (AHEI), 7-dimethylaminonaphthalene-1,2-dicarboxylic acid hydrazide, ring-substituted aminophthalhydrazides, anthracene-2,3-dicarboxylic acid hydrazides, phenanthrene-1,2-dicarboxylic acid hydrazides, pyrenedicarboxylic acid hydrazides, 5-hydroxyphthal-hydrazide, 6-hydroxyphthalhydrazide, as well as other phthalazinedione analogs disclosed in U.S. Pat. No. 5,420,275 to Masuya et al. and in U.S. Pat. No. 5,324,835 to Yamaguchi. Other examples for compounds that may serve as a chemiluminescent moiety of the chemiluminescent compound used in the present invention are xanthene dyes such as fluorescein, eosin, rhodamine dyes, or rhodol dyes, aromatic amines and heterocyclic amines, acridan esters, thioesters and sulfonamides, and acridan ketenedithioacetal compounds that are known in the art to produce chemiluminescence by reaction with peroxide and peroxidase.

The term “alkyl,” by itself or as part of another substituent, means, unless otherwise stated, a straight or branched chain, or cyclic hydrocarbon radical, or combination thereof, which may be fully saturated, mono- or polyunsaturated and can include di- and multivalent radicals, having the number of carbon atoms designated (i.e. C₁-C₁₀ means one to ten carbons). Examples of saturated hydrocarbon radicals include, but are not limited to, groups such as methyl, ethyl, n-propyl, isopropyl, n-butyl, t-butyl, isobutyl, sec-butyl, cyclohexyl, (cyclohexyl)methyl, cyclopropylmethyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. The term “alkyl,” unless otherwise noted, is also meant to include those derivatives of alkyl defined in more detail below, such as “heteroalkyl.” Alkyl groups that are limited to hydrocarbon groups are termed “homoalkyl”. An alkoxy is an alkyl attached to the remainder of the molecule via an oxygen linker (—O—).

The term “alkylene” by itself or as part of another substituent means a divalent radical derived from an alkyl, as exemplified, but not limited, by —CH₂CH₂CH₂CH₂—, and further includes those groups described below as “heteroalkylene.” Typically, an alkyl (or alkylene) group will have from 1 to 24 carbon atoms, with those groups having 10 or fewer carbon atoms being preferred in the present invention. A “lower alkyl” or “lower alkylene” is a shorter chain alkyl or alkylene group, generally having eight or fewer carbon atoms.

The term “heteroalkyl,” by itself or in combination with another term, means, unless otherwise stated, a stable straight or branched chain, or cyclic hydrocarbon radical, or combinations thereof, consisting of at least one carbon atoms and at least one heteroatom selected from the group consisting of O, N, P, Si and S, and wherein the nitrogen and sulfur atoms may optionally be oxidized and the nitrogen heteroatom may optionally be quaternized. The heteroatom(s) O, N, P and S and Si may be placed at any interior position of the heteroalkyl group or at the position at which the alkyl group is attached to the remainder of the molecule. Examples include, but are not limited to, —CH₂—CH₂—O—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CH—O—CH₃, —Si(CH₃)₃, —CH₂—CH═N—OCH₃, —CH═CH—N(CH₃)—CH₃, O—CH₃, —O—CH₂—CH₃, and —CN. Up to two heteroatoms may be consecutive, such as, for example, —CH₂—NH—OCH₃. Similarly, the term “heteroalkylene” by itself or as part of another substituent means a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′— represents both —C(O)₂R′— and —R′C(O)₂—. As described above, heteroalkyl groups, as used herein, include those groups that are attached to the remainder of the molecule through a heteroatom, such as —C(O)R′, —C(O)NR′, —NR′R″, —OR′, —SR′, and/or —SO₂R′. Where “heteroalkyl” is recited, followed by recitations of specific heteroalkyl groups, such as —NR′R″ or the like, it will be understood that the terms heteroalkyl and —NR′R″ are not redundant or mutually exclusive. Rather, the specific heteroalkyl groups are recited to add clarity. Thus, the term “heteroalkyl” should not be interpreted herein as excluding specific heteroalkyl groups, such as —NR′R″ or the like.

The terms “cycloalkyl” and “heterocycloalkyl” (also referred to herein as a “heterocyclic”), by themselves or in combination with other terms, represent, unless otherwise stated, cyclic versions of “alkyl” and “heteroalkyl”, respectively. Additionally, for heterocycloalkyl, a heteroatom can occupy the position at which the heterocycle is attached to the remainder of the molecule. Examples of cycloalkyl include, but are not limited to, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like. The terms “cycloalkylene” and “heterocycloalkylene” refer to the divalent derivatives of cycloalkyl and heterocycloalkyl, respectively.

The terms “halo” or “halogen,” by themselves or as part of another substituent, mean, unless otherwise stated, a fluorine, chlorine, bromine, or iodine atom. Additionally, terms such as “haloalkyl,” are meant to include monohaloalkyl and polyhaloalkyl. For example, the term “halo(C₁-C₄)alkyl” is mean to include, but not be limited to, trifluoromethyl, 2,2,2-trifluoroethyl, 4-chlorobutyl, 3-bromopropyl, and the like.

The term “acyl” means —C(O)R where R is a substituted or unsubstituted alkyl, substituted or unsubstituted cycloalkyl, substituted or unsubstituted heteroalkyl, substituted or unsubstituted heterocycloalkyl, substituted or unsubstituted aryl or substituted or unsubstituted heteroaryl.

The term “aryl” means, unless otherwise stated, a polyunsaturated, aromatic, substituent that can be a single ring or multiple rings (preferably from 1 to 3 rings), which are fused together or linked covalently. The term “heteroaryl” refers to aryl groups (or rings) that contain from one to four heteroatoms selected from N, O, and S, wherein the nitrogen and sulfur atoms are optionally oxidized, and the nitrogen atom(s) are optionally quaternized. A heteroaryl group can be attached to the remainder of the molecule through a heteroatom. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below. The terms “arylene” and “heteroarylene” refer to the divalent radicals of aryl and heteroaryl, respectively.

For brevity, the term “aryl” when used in combination with other terms (e.g., aryloxy, arylthioxy, arylalkyl) includes both aryl and heteroaryl rings as defined above. Thus, the term “arylalkyl” is meant to include those radicals in which an aryl group is attached to an alkyl group (e.g., benzyl, phenethyl, pyridylmethyl and the like) including those alkyl groups in which a carbon atom (e.g., a methylene group) has been replaced by, for example, an oxygen atom (e.g., phenoxymethyl, 2-pyridyloxymethyl, 3-(1-naphthyloxy)propyl, and the like).

The term “oxo” as used herein means an oxygen that is double bonded to a carbon atom.

The term “alkylsulfonyl” as used herein means a moiety having the formula —S(O₂)—R′, where R′ is an alkyl group as defined above. R′ may have a specified number of carbons (e.g. “C₁-C₄ alkylsulfonyl”).

Each of the above terms (e.g., “alkyl,” “heteroalkyl,” “aryl” and “heteroaryl”) are meant to include both substituted and unsubstituted forms of the indicated radical. Preferred substituents for each type of radical are provided below.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) are generically referred to as “alkyl group substituents,” and they can be one or more of a variety of groups selected from, but not limited to: —OR′, ═O, ═NR′, ═N—OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR″—C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —CN, —⁺NR₃, —⁺PR₃, —B(OH)₂, and —NO₂ in a number ranging from zero to (2 m′+1), where m′ is the total number of carbon atoms in such radical. R′, R″, R′″ and R″″ each preferably independently refer to hydrogen, substituted or unsubstituted heteroalkyl, substituted or unsubstituted aryl, e.g., aryl substituted with 1-3 halogens, substituted or unsubstituted alkyl, alkoxy or thioalkoxy groups, or arylalkyl groups. When a compound of the invention includes more than one R group, for example, each of the R groups is independently selected as are each R′, R″, R′″ and R″″ groups when more than one of these groups is present. When R′ and R″ are attached to the same nitrogen atom, they can be combined with the nitrogen atom to form a 5-, 6-, or 7-membered ring. For example, —NR′R″ is meant to include, but not be limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for the alkyl radical, substituents for the aryl and heteroaryl groups are generically referred to as “aryl group substituents.” The substituents are selected from, for example: halogen, —OR′, ═O, ═NR′, ═N—OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —CN, —⁺NR₃, —⁺PR₃, —B(OH)₂, and —NO₂, —R′, —N₃, —CH(Ph)₂, fluoro(C₁-C₄)alkoxy, and fluoro(C₁-C₄)alkyl, in a number ranging from zero to the total number of open valences on the aromatic ring system; and where R′, R″, R′″ and R″″ are preferably independently selected from hydrogen, substituted or unsubstituted alkyl, substituted or unsubstituted heteroalkyl, substituted or unsubstituted aryl and substituted or unsubstituted heteroaryl. When a compound of the invention includes more than one R group, for example, each of the R groups is independently selected as are each R′, R″, R′″ and R″″ groups when more than one of these groups is present.

Two of the substituents on adjacent atoms of the aryl or heteroaryl ring may optionally be replaced with a substituent of the formula -T-C(O)—(CRR′)_{q}—U—, wherein T and U are independently —NR—, —O—, —CRR′— or a single bond, and q is an integer of from 0 to 3. Alternatively, two of the substituents on adjacent atoms of the aryl or heteroaryl ring may optionally be replaced with a substituent of the formula -A-(CH₂)ᵣ—B—, wherein A and B are independently —CRR′—, —O—, —NR—, —S—, —S(O)—, —S(O)₂—, —S(O)₂NR′— or a single bond, and r is an integer of from 1 to 4. One of the single bonds of the new ring so formed may optionally be replaced with a double bond. Alternatively, two of the substituents on adjacent atoms of the aryl or heteroaryl ring may optionally be replaced with a substituent of the formula —(CRR′)ₛ—X—(CR″R′″)_{d}—, where s and d are independently integers of from 0 to 3, and X is —O—, —NR′—, —S—, —S(O)—, —S(O)₂—, or —S(O)₂NR′—. The substituents R, R′, R″ and R′″ are preferably independently selected from hydrogen or substituted or unsubstituted (C₁-C₆)alkyl.

As used herein, the term “heteroatom” is meant to include oxygen (O), nitrogen (N), sulfur (S), silicon (Si) and phosphorus (P).

A “substituent group,” as used herein, means a group selected from the following moieties:

(A) —OH, —NH₂, —SH, —CN, —CF₃, —NO₂, oxo, halogen, unsubstituted alkyl, unsubstituted heteroalkyl, unsubstituted cycloalkyl, unsubstituted heterocycloalkyl, unsubstituted aryl, unsubstituted heteroaryl, and

(B) alkyl, heteroalkyl, cycloalkyl, heterocycloalkyl, aryl, and heteroaryl, substituted with at least one substituent selected from:

(i) oxo, —OH, —NH₂, —SH, —CN, —CF₃, —⁺NR₃, —⁺PR₃, —B(OH)₂, —NO₂, halogen, unsubstituted alkyl, unsubstituted heteroalkyl, unsubstituted cycloalkyl, unsubstituted heterocycloalkyl, unsubstituted aryl, unsubstituted heteroaryl, and

(ii) alkyl, heteroalkyl, cycloalkyl, heterocycloalkyl, aryl, and heteroaryl, substituted with at least one substituent selected from:

(a) oxo, —OH, —NH₂, —SH, —CN, —CF₃, —NO₂, halogen, unsubstituted alkyl, unsubstituted heteroalkyl, unsubstituted cycloalkyl, unsubstituted heterocycloalkyl, unsubstituted aryl, unsubstituted heteroaryl, and

(b) alkyl, heteroalkyl, cycloalkyl, heterocycloalkyl, aryl, or heteroaryl, substituted with at least one substituent selected from oxo, —OH, —NH₂, —SH, —CN, —CF₃, —NO₂, halogen, unsubstituted alkyl, unsubstituted heteroalkyl, unsubstituted cycloalkyl, unsubstituted heterocycloalkyl, unsubstituted aryl, and unsubstituted heteroaryl.

As used herein, “amino acid” refers to a group of water-soluble compounds that possess both a carboxyl and an amino group attached to the same carbon atom. Amino acids can be represented by the general formula NH₂—CHR—COOH where R may be hydrogen or an organic group, which may be nonpolar, basic acidic, or polar. As used herein, “amino acid” refers to both the amino acid radical and the non-radical free amino acid.

The term “hydroxy” is used herein to refer to the group —OH.

The term “amino” is used to describe primary amines, —NRR′, wherein R and R′ are independently H, alkyl, aryl or substituted analogues thereof “Amino” encompasses “alkylamino” denoting secondary and tertiary amines and “acylamino” describing the group RC(O)NR′.

The term “alkoxy” is used herein to refer to the —OR group, where R is alkyl, aryl, or substituted analogues thereof. Suitable alkoxy radicals include, for example, methoxy, ethoxy, phenoxy, substituted phenoxy, benzyloxy, phenethyloxy, t-butoxy, etc.

The term “acyloxy” is used herein to describe an organic radical derived from an organic acid by the removal of the acidic hydrogen. Simple acyloxy groups include, for example, acetoxy, and higher homologues derived from carboxylic acids such as ethanoic, propanoic, butanoic, etc. The acyloxy moiety may be oriented as either a forward or reverse ester (i.e. RC(O)OR′ or R′OC(O)R).

A “ring,” as used herein, refers to a substituted or unsubstituted cycloalkyl, substituted or unsubstituted heterocycloalkyl, substituted or unsubstituted aryl, and/or substituted or unsubstituted heteroaryl.

As used herein, “nucleic acid” means either DNA, RNA, single-stranded, double-stranded, or more highly aggregated hybridization motifs, and any chemical modifications thereof. Modifications include, but are not limited to, those which provide other chemical groups that incorporate additional charge, polarizability, hydrogen bonding, electrostatic interaction, and functionality to the nucleic acid ligand bases or to the nucleic acid ligand as a whole. Such modifications include, but are not limited to, peptide nucleic acids, phosphodiester group modifications (e.g., phosphorothioates, methylphosphonates), 2′-position sugar modifications, 5-position pyrimidine modifications, 8-position purine modifications, modifications at exocyclic amines, substitution of 4-thiouridine, substitution of 5-bromo or 5-iodo-uracil; backbone modifications, methylations, unusual base-pairing combinations such as the isobases isocytidine and isoguanidine and the like. Modifications can also include 3′ and 5′ modifications such as capping.

A “nucleobase” is a nucleoside or nucleotide. A “nucleoside” is a deoxyribose or ribose sugar, or derivative thereof, containing a nitrogenous base linked to the C1′ of the sugar residue. A “nucleotide” is the C5′ phosphate ester derivative of a nucleoside. The terms “nucleoside and “nucleotide” include those compounds having non-natural substituents at the C1′, C2′, C3′, C5′, and/or nitrogenous base (e.g., C2′ alkyl, alkoxy, and halogen substituents).

“Polypeptide” refers to a polymer in which the monomers are amino acids and are joined together through amide bonds, alternatively referred to as a peptide. When the amino acids are α-amino acids, either the l-optical isomer or the d-optical isomer can be used. Additionally, unnatural amino acids, for example, β-alanine, phenylglycine and homoarginine are also included. Commonly encountered amino acids that are not gene-encoded may also be used in the present invention. All of the amino acids used in the present invention may be either the d- or 1-isomer. The 1-isomers are generally preferred. In addition, other peptidomimetics are also useful in the present invention. For a general review, see, Spatola, A. F., in Chemistry and Biochemistry of Amino Acids, Peptides and Proteins, B. Weinstein, eds., Marcel Dekker, New York, p. 267 (1983).

### II. Methods of Analyte Detection

In one aspect, a novel method of detecting an analyte in a sample is provided. The method includes contacting the analyte with a solid support conjugate and a first analyte binder conjugate. The first analyte binder conjugate is a peroxidase enzyme conjugated to a first analyte binder. The solid support conjugate is a solid support that is conjugated to a second analyte binder, a peroxide generating enzyme, and a chemiluminescent compound. The analyte is allowed to bind to the first analyte binder and the second analyte binder thereby forming a detectable solid support bound analyte complex. The detectable solid support bound analyte complex is contacted with a peroxide generating enzyme substrate thereby producing a peroxide. The peroxidase enzyme is allowed to react with the peroxide which results in the activation of the chemiluminescent compound and the production of a chemiluminescent signal. The analyte is detected by detecting the chemiluminescent signal. In some embodiments, the detectable solid support bound analyte complex is also contacted with a peroxidase enhancer compound.

Analytes that are detected using the methods provided herein include, cardiac markers and cardiac drugs such as Troponin I, CK-MB, digoxin, myoglobin and BNP. In other embodiments, the analyte is a drug and analyte related to reproductive function including AFP, DHEA-S, estradiol, FSH, LK, Inhibin A, PAPP-A, PIGF, sVEGF R1, progesterone, prolactin, SHBG, testosterone, βHCG, and unconjugated estriol. Other analytes include indicators of and drugs for treatment of anemia including EPO, ferritin, folate, Intrinsic Factor Ab, soluble transferrin receptor, and vitamin B12. Other analytes include intact PTH, bone alkaline phosphatase, and hGH for assessing bone metabolism. In some embodiments, analytes for assessing thyroid function include free and total T3, free and total T4, TSH, thyroglobulin, thyroglobulin Ab and TPO Ab. Other analytes include tumor markers AFP, BPHA, CA 15-3 antigen, CEA, CA 19-9 antigen, PSA, and CA 125 antigen. Infectious disease analytes include CMV IgG and IgM, Rubella IgG and IgM, Toxoplasmosis IgG and IgM, HAV Ab and IgM, HBc Ab and IgM, Hbe Ab and Antigen, HBs Ab and Antigen, and HCV Ab.

Any appropriate peroxidase enzyme may be used in the methods provided herein. Peroxidase enzymes reduce hydrogen peroxide to water while oxidizing a variety of substrates. Exemplary peroxidase enzymes include a horseradish peroxidase enzyme, a peanut peroxidase enzyme, a barley grain peroxidase enzyme, an ascorbate peroxidase enzyme, a fungal peroxidase or a cytochrome-C peroxidase enzyme. In some embodiments, the peroxidase enzyme is a horseradish peroxidase.

The choice of solid support for use in the present methods is based upon the desired assay format and performance characteristics. Acceptable solid supports for use in the present methods can vary widely. A solid support can be porous or nonporous. It can be continuous or non-continuous, and flexible or nonflexible. A solid support can be made of a variety of materials including ceramic, glass, metal, organic polymeric materials, or combinations thereof. Moreover, the solid support provided herein may be a magnetic solid support. The magnetic solid support may be composed at least in part of a magnetically responsive component such as a magnetic particle. Magnetic particles can have a solid core portion that is magnetically responsive and is surrounded by one or more non-magnetically responsive layers. Magnetically responsive components include magnetically responsive materials such as ferromagnetic, paramagnetic and superparamagnetic materials. One exemplary magnetically responsive material is magnetite.

The solid support may further be coated with one or more coating particles. Such coating particles may function to provide reactive groups to conjugate the chemiluminescent moiety to the solid support. The chemiluminescent moiety may be connected to the solid support by reacting a reactive group of the linking moiety with a reactive group of the solid support. Reactive groups are further discussed below. In some embodiments, the coating particles may include BSA providing sulfhydryl, amino or carboxyl groups as reactive groups. In certain embodiments, the coating particles form at least part of a coating layer on the solid support.

A peroxide-generating enzyme is an enzyme that catalyzes the oxidation or reduction reaction of a variety of substrates involving molecular oxygen as the electron acceptor. In such reactions oxygen is reduced to hydrogen peroxide or a combination of water and hydrogen peroxide. The generated hydrogen peroxide is then reduced to water by the peroxidase enzyme in the present reaction system. Examples of peroxide generating enzymes used in the embodiments presented include, but are not limited to, glucose oxidase, glycollate oxidase, hexose oxidase, cholesterol oxidase, aryl-alcohol oxidase, L-gulonolactone oxidase, galactose oxidase, pyranose oxidase, L-sorbose oxidase, pyridoxine oxidase, alcohol oxidase, L-2-hydroxy-acid oxidase, ecdysome oxidase, choline oxidase, aldehyde oxidase, xanthine oxidase, pyruvate oxidase, oxalate oxidase, glyoxylate oxidase, pyruvate oxidase, D-aspartate oxidase, L-aminoacid oxidase, amine oxidase, pyridoxaminephosphate oxidase, D-glutamate oxidase, ethanolamine oxidase, tyramine oxidase, putrascine oxidase, sarcosine oxidase, N-methylaminoacid oxidase, N-methyl-lysine oxidase, hydroxylnicotine oxidase, nitroethane oxidase, acetyl-indoxyl oxidase, urate oxidase, hydroxylamine oxidase, or sulphite oxidase. Any appropriate peroxide generating enzyme substrate may be used during the reduction or oxidation reaction catalyzed by the peroxidase generating enzyme. Examples for peroxide generating enzyme substrates are glucose, glycollate, hexose, cholesterol, aryl-alcohol, L-gulonolactone, galactose, pyranose, L-sorbose, pyridoxine, alcohol, L-2-hydroxy-acid, ecdysome, choline, aldehyde, xanthine, pyruvate, oxalate, glyoxylate, pyruvate, D-aspartate, L-aminoacid, amine, pyridoxaminephosphate, D-glutamate, ethanolamine, tyramine, putrascine, sarcosine, N-methylaminoacid, N-methyl-lysine, hydroxylnicotine, nitroethane, acetyl-indoxyl, urate, hydroxylamine, or sulphite. The reaction of a peroxide generating enzyme with a corresponding peroxide generating enzyme substrate results in oxidation or reduction of the peroxide generating enzyme substrate and production of hydrogen peroxide due to the reduction of oxygen. One of skill will immediately identify the corresponding substrates and enzymes listed above (e.g. the substrate for oxalate oxidase is oxalate). In some embodiments, glucose oxidase may be used as the peroxide generating enzyme to react with glucose as the peroxide generating enzyme substrate thereby reducing oxygen to hydrogen peroxide. Hydrogen peroxide may then be reduced to water by a peroxidase enzyme. Therefore, in some embodiments, the peroxide generating enzyme is glucose oxidase and the peroxide generating enzyme substrate is glucose.

As described above, in some embodiments, the detectable solid support bound analyte complex is contacted with a peroxidase enhancer compound. Typically the peroxidase enhancer compound is present when the detectable solid support bound analyte complex is contacted with the peroxide generating enzyme substrate thereby producing peroxide. Without being limited by any particular mechanistic theory, it is believed that an oxidized peroxidase enhancer compound is generated from a peroxidase enhancer compound when the peroxidase enzyme reacts with hydrogen peroxide. The oxidized peroxidase enhancer compound may promote the catalytic activity of the peroxidase with a chemiluminescent compound during the process of generating luminescence. Thus, peroxidase enhancer compounds may be contacted with a detectable solid support bound analyte complex when the peroxide generating enzyme substrate is added. In the methods described herein, a peroxidase enhancer compound may include a phenolic moiety. In some embodiments, the peroxidase enhancer may be p-phenylphenol, p-iodophenol, p-bromophenol, p-hydroxycinnamic acid, p-imidazolylphenol, acetaminophen, 2,3,-dichlorophenol, 2-naphthol, or 6-bromo-2-naphthol or other art-known enhancers. Included among the enhancers for use herein are phenolic compounds and aromatic amines known to enhance other peroxidase reactions as described in U.S. Pat. Nos. 5,171,668 and 5,206,149. Substituted and unsubstituted arylboronic acid compounds and their ester and anhydride derivatives as disclosed in U.S. Pat. No. 5,512,451 are another class of compounds considered to be within the scope of enhancers useful in the present methods. Derivatives of phenoxazine and phenothiazine including 3-(N-phenothiazinyl)-propanesulfonic acid salts, 3-(N-phenoxazinyl)propanesulfonic acid salts, 4-(N-phenoxazinyl)butanesulfonic acid salts, 5-(N-phenoxazinyl)-pentanoic acid salts and N-methylphenoxazine and related homologs represent another useful group of enhancer compounds.

The first analyte binder and the second analyte binder may be binding proteins such as, but not limited to, antibodies, antibody fragments, antibody-DNA chimeras, antigens, haptens, peptides, hormone receptors, protein A, lectin, avidin, streptavidin and biotin. In some embodiments, the first analyte binder and the second analyte binder are binding proteins. In other embodiment, the first analyte binder and the second analyte binder are antibodies.

Again, without being limited by any particular mechanistic theory, it is believed that chemiluminescence is the emission of light as the result of a chemical reaction. In the presence of a suitable catalyst a chemiluminescent compound may be transferred into a higher state of energy due to the transfer of energy from a second reaction partner. The decay of the excited state of the chemiluminescent compound to a lower energy level may result in the emission of light. Upon relaxation to a ground state the chemiluminescent compound may either directly emit light or may transfer the excitation energy to an emissive energy acceptor, which is the source of light emission. The chemiluminescent compound useful herein typically comprises a chemiluminescent moiety, which may be transferred into a higher state of energy and a linking moiety for coupling to another material. The chemiluminescent moiety includes each class of compounds described above including, but not limited to, luminal and structurally related cyclic hydrazides, acridan esters, thioesters and sulfonamides, and acridan ketenedithioacetal compounds. In some embodiments, the chemiluminescent compound includes a chemiluminescent acridan moiety. Acridans represent compounds that react either directly or indirectly with a peroxidase enzyme and/or peroxide to produce a chemiluminescent signal. The following patents disclose chemiluminescent acridan moietie useful in the methods provided herein: U.S. Pat. Nos. 5,491,072, 5,523,212, 5,593,845, 5,750,698, 6,858,733, 6,872,828 and 7,247,726.

During the process of hydrogen peroxide decomposition water and oxygen are produced either spontaneously or due to the presence of a decomposition agent. Decomposition agents catalyze the decomposition of peroxide to water and oxygen thereby removing excess peroxide from the reaction. Thus, background signal is reduced during analysis involving use of peroxidase conjugated analyte binders. The detectable solid support bound analyte complex may contacted with a peroxide decomposition agent for background signal reducing purposes. Therefore, in some embodiments, the detectable solid support bound analyte complex is contacted with a peroxide decomposition agent. In other embodiments, the detectable solid support bound analyte complex is contacted with the peroxide decomposition agent before being contacted with the peroxide generating enzyme substrate and the production of peroxide. The decomposition agents provided herein may be aromatic hydrocarbons or their derivatives. The decomposition agent may also be an enzyme that is able to react with hydrogen peroxide to produce water and oxygen. In some embodiments, the decomposition agent is an enzyme. In other embodiments, the decomposition agent is a catalase. In some embodiments, the catalase is present with the detectable solid support bound analyte complex at concentrations between 0.1 to 10 μg/ml. In other embodiments, the catalase is present with the detectable solid support bound analyte complex at concentrations between 0.5 to 5 μg/ml. In other embodiments, the catalase is present with the detectable solid support bound analyte complex at a concentration of about 2 μg/ml (e.g. 2 μg/ml). The catalase enzyme may be derived from prokaryotic or eukaryotic cells. In some examples, catalase enzymes are derived from human erythrocytes. Further, the catalase enzyme may be derived from murine, bovine or bison liver.

It is sometimes desirable to detect an analyte in a sample using competition binding assays. During such competition binding assays the first or second analyte binder (which is conjugated to a solid support conjugate) interacts with a competition analyte. A competition analyte is a binding partner able to interact with the first or second analyte binder. The term competition analyte refers to, but is not limited to, a binding partner such as a protein, peptide or antibody that is able to interact with the first or second analyte binder. Among other things, the competition analyte may be a carbohydrate, peptide, protein, nucleic acid or drug (e.g. a hormone, cytokine, enzyme substrate, viruses, biomolecules, or small molecule modulator). In some embodiments, the competition analyte is a purified form of an analyte found in nature or a synthetic version of the analyte (e.g. an analyte produced chemically or using recombinant techniques). In other embodiments, the competition analyte is a competition analyte analog. A competition analyte analog is a binding partner with properties that enable the competition analyte analog to compete with the analyte for interaction with the first or second analyte binder. Examples of competition analyte analogs are nucleic acid analogs such as peptide nucleic acid (PNA) or conjugated polymers with DNA-mimetic properties, nonnatural and natural peptide analogs, peptide mimetics that biologically mimic active determinants on hormones, cytokines, enzyme substrates, viruses or other bio-molecules, and small molecule modulators (such as those having high affinity to the ATP binding site of ATP-dependent enzymes).

Subsequent to binding the first or second analyte binder, the competition analyte competes with the analyte for interaction with the second or first analyte binder, respectively. A detectable solid support bound analyte complex may be formed upon binding of the competition analyte to the first analyte binder and the second analyte binder. However, if the analyte is bound to either the first or the second analyte binder within this competition binding assay, a detectable solid support bound analyte complex may be prevented from forming. Therefore, in the competition binding assays presented herein, lower amounts of analyte result in a stronger chemiluminescent signal, whereas higher concentrations of analyte result in a weaker chemiluminescent signal.

In one aspect, the method includes contacting the analyte with a solid support conjugate and a first analyte binder conjugate. The first analyte binder conjugate is a peroxidase enzyme conjugated to a first analyte binder where the first analyte binder is bound to a competition analyte. The solid support conjugate includes a solid support conjugated to a second analyte binder, a peroxide generating enzyme, and a chemiluminescent compound. The binding of the first analyte binder conjugate, which includes the competition analyte, to the second analyte binder forms a detectable solid support bound analyte complex. The detectable solid support bound analyte complex is contacted with a peroxide generating enzyme substrate thereby producing a peroxide. The peroxidase enzyme is allowed to react with the peroxide which results in the activation of the chemiluminescent compound and the production of a chemiluminescent signal. The analyte is detected by detecting the chemiluminescent signal insofar as the amount of analyte correlates inversely to the intensity of the chemiluminescent signal. Thus, detecting the chemiluminescent signal may include detecting a lower amount of chemiluminescent signal or absence of chemiluminescent signal.

In another embodiment, the method includes contacting the analyte with a solid support conjugate and a first analyte binder conjugate. The first analyte binder conjugate is a peroxidase enzyme conjugated to a first analyte binder. The solid support conjugate includes a solid support conjugated to a peroxide generating enzyme, a chemiluminescent compound and a second analyte binder where the second analyte binder is bound to a competition analyte. The binding of the first analyte binder conjugate to the solid support conjugate, which includes the competition analyte, forms a detectable solid support bound analyte complex. The detectable solid support bound analyte complex is contacted with a peroxide generating enzyme substrate thereby producing a peroxide. The peroxidase enzyme is allowed to react with the peroxide which results in the activation of the chemiluminescent compound and the production of a chemiluminescent signal. The analyte is detected by detecting the chemiluminescent signal insofar as the amount of analyte correlates inversely to the intensity of the chemiluminescent signal. Thus, detecting the chemiluminescent signal may include detecting a lower amount of chemiluminescent signal or absence of chemiluminescent signal.

In one aspect, the method includes contacting the analyte, or a sample including the analyte, with a solid support conjugate and a first analyte binder conjugate. The first analyte binder conjugate is a peroxidase enzyme conjugated (e.g. covalently bound) to a competition analyte. The competition analyte may be directly conjugated to the peroxidase enzyme or linked through a bifunctional linker. The solid support conjugate includes a solid support conjugated to a peroxide generating enzyme, a chemiluminescent compound and a second analyte binder. The binding of the first analyte binder conjugate, which includes the competition analyte conjugated to the peroxidase enzyme, to the solid support conjugate forms a detectable solid support bound analyte complex. The detectable solid support bound analyte complex is contacted with a peroxide generating enzyme substrate thereby producing a peroxide. The peroxidase enzyme is allowed to react with the peroxide which results in the activation of the chemiluminescent compound and the production of a chemiluminescent signal. The analyte is detected by detecting the chemiluminescent signal insofar as the amount of analyte correlates inversely to the intensity of the chemiluminescent signal. Thus, detecting the chemiluminescent signal may include detecting a lower amount of chemiluminescent signal or absence of chemiluminescent signal.

In another aspect, the method includes contacting the analyte with a solid support conjugate and a first analyte binder conjugate. The first analyte binder conjugate is a peroxidase enzyme conjugated to a first analyte binder. The solid support conjugate includes a competition solid support conjugate which includes a solid support conjugated to a peroxide generating enzyme, a chemiluminescent compound, and a competition analyte that is covalently bound to the solid support. The competition analyte may be directly conjugated to the solid support or through a bifunctional linker. The binding of the first analyte binder conjugate to the competition solid support conjugate forms a detectable solid support bound analyte complex. The detectable solid support bound analyte complex is contacted with a peroxide generating enzyme substrate thereby producing a peroxide. The peroxidase enzyme is allowed to react with the peroxide which results in the activation of the chemiluminescent compound and the production of a chemiluminescent signal. The analyte is detected by detecting the chemiluminescent signal insofar as the amount of analyte correlates inversely to the intensity of the chemiluminescent signal. Thus, detecting the chemiluminescent signal may include detecting a lower amount of chemiluminescent signal or absence of chemiluminescent signal.

In another aspect, the method includes contacting the analyte with a solid support conjugate and an analyte-peroxidase conjugate. The analyte-peroxidase conjugate is a peroxidase enzyme conjugated to the analyte or a homolog of the analyte. The solid support conjugate includes a solid support conjugated to a peroxide generating enzyme, a chemiluminescent compound, and a first analyte binder that is covalently bound to the solid support. The first analyte binder may be directly conjugated to the solid support or through a bifunctional linker. The binding of the first analyte binder to the analyte-peroxidase conjugate forms a detectable solid support bound analyte complex. The detectable solid support bound analyte complex is contacted with a peroxide generating enzyme substrate thereby producing a peroxide. The peroxidase enzyme is allowed to react with the peroxide which results in the activation of the chemiluminescent compound and the production of a chemiluminescent signal. The analyte is detected by detecting the chemiluminescent signal insofar as the amount of analyte correlates inversely to the intensity of the chemiluminescent signal. Thus, detecting the chemiluminescent signal may include detecting a lower amount of chemiluminescent signal or absence of chemiluminescent signal.

In one aspect, a solid support conjugate is provided and the solid support conjugate includes a solid support conjugated to a chemiluminescent compound, a hydrogen peroxide generating enzyme, and an analyte binder. In some embodiments, the analyte binder is an antibody. In other embodiments, the hydrogen peroxide generating enzyme is a glucose oxidase. In other embodiments, the chemiluminescent compound includes a chemiluminescent acridan moiety. In some embodiments, the chemiluminescent compound has the formula:

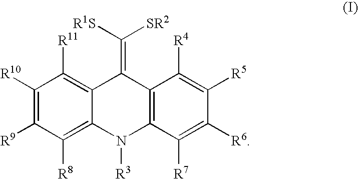

R¹ and R² may independently be substituted or unsubstituted alkyl, substituted or unsubstituted alkenyl, substituted or unsubstituted alkynyl, substituted or unsubstituted aryl, or substituted or unsubstituted aralkyl. R³ is substituted or unsubstituted alkyl, substituted or unsubstituted alkenyl, substituted or unsubstituted alkynyl, substituted or unsubstituted aryl, substituted or unsubstituted aralkyl, alkoxyalkyl, carboxyalkyl or alkylsulfonic acid. R³ is optionally joined with R⁷ or R⁸ to form a 5 or 6-membered ring. R⁴, R⁵, R⁶, R⁷, R⁸, R⁹, R¹⁰, and R¹¹ are independently hydrogen, substituted or unsubstituted alkyl, substituted or unsubstituted aryl, aralkyl, alkenyl, alkynyl, alkoxy, aryloxy, halogen, amino, substituted amino, substituted or unsubstituted carboalkoxy, carboxamide, cyano, or sulfonate. Pairs of adjacent groups of R⁴, R⁵, R⁶, R⁷, R⁸, R⁹, R¹⁰, and R¹¹ are optionally joined to form a carbocyclic or heterocyclic ring system. At least one of the groups of R¹ to R¹¹ includes a linking moiety. In one embodiment, each of R⁴ to R¹¹ is H.

R¹ and R² in the compound of formula I can be any organic group containing from 1 to about 50 non hydrogen atoms selected from C, N, O, S, P, Si and halogen atoms which allows light production. By the latter is meant that when a compound of formula I undergoes a reaction of set forth in the methods provided herein, an excited state product compound is produced and can involve the production of one or more chemiluminescent intermediates. The excited state product can emit the light directly or can transfer the excitation energy to a fluorescent acceptor through energy transfer causing light to be emitted from the fluorescent acceptor. In one embodiment R¹ and R² are selected from substituted or unsubstituted alkyl, substituted or unsubstituted alkenyl, substituted or unsubstituted alkynyl, substituted or unsubstituted aryl, and substituted or unsubstituted aralkyl groups of 1-20 carbon atoms. When R¹ or R² are substituted, it can be substituted with 1-3 groups selected from carbonyl groups, carboxyl groups, tri(C₁-C₈ alkyl)silyl groups, a SO₃⁻ group, a OSO₃⁻² group, glycosyl groups, a PO₃⁻ group, a OPO₃⁻² group, halogen atoms, a hydroxyl group, a thiol group, amino groups, quaternary ammonium groups, and quaternary phosphonium groups.

R³ is an organic group containing from 1 to 50 non-hydrogen atoms selected from C, N, O, S, P, Si and halogen in addition to the necessary number of H atoms required to satisfy the valences of the atoms in the group. In one embodiment, R³ contains from 1 to 20 non-hydrogen atoms. In another embodiment, the organic group is selected from the group consisting of alkyl, substituted alkyl, substituted or unsubstituted alkenyl, substituted or unsubstituted alkynyl, substituted or unsubstituted aryl, and substituted or unsubstituted aralkyl groups of 1 to 20 carbon atoms. In another embodiment, R³ includes substituted or unsubstituted C₁-C₄ alkyl groups, phenyl, substituted or unsubstituted benzyl groups, alkoxyalkyl, carboxyalkyl and alkylsulfonic acid groups. R³ can be joined to either R⁷ or R⁸ to complete a 5 or 6-membered ring. In one embodiment, R³ is substituted with a linking moiety.

In the compounds of Formula (I), R⁴ to R¹¹ each are independently H or a substituent which permits the excited state product to be produced and generally contain from 1 to 50 atoms selected from C, N, O, S, P, Si and halogens. Representative substituents which can be present include, without limitation, alkyl, substituted alkyl, aryl, substituted aryl, aralkyl, alkenyl, alkynyl, alkoxy, aryloxy, halogen, amino, substituted amino, carboxyl, carboalkoxy, carboxamide, cyano, and sulfonate groups. Pairs of adjacent groups, e.g., R⁴ to R⁵ or R⁵ to R⁶, can be joined together to form a carbocyclic or heterocyclic ring system comprising at least one 5 or 6-membered ring which is fused to the ring to which the two groups are attached. Such fused heterocyclic rings can contain N, O or S atoms and can contain ring substituents other than H such as those mentioned above. One or more of the groups R⁴ to R¹¹ can be a linking moiety. In one embodiment, R⁴ to R¹¹ are selected from hydrogen, halogen and alkoxy groups such as methoxy, ethoxy, t-butoxy and the like. In another embodiment, a group of compounds has one of R⁵, R⁶, R⁹ or R¹⁰ as a halogen and the other of R⁴ to R¹¹ are hydrogen atoms.

Substituents can be incorporated in various quantities and at selected ring or chain positions in the acridan ring in order to modify the properties of the chemiluminescent compound or to provide for convenience of synthesis. Such properties include, e.g., chemiluminescence quantum yield, rate of reaction with the enzyme, maximum light intensity, duration of light emission, wavelength of light emission and solubility in the reaction medium. Specific substituents and their effects are illustrated in the specific examples below, which, however, are not to be considered limiting the scope of the invention in any way. For synthetic expediency compounds of formula I desirably have each of R⁴ to R¹¹ as a hydrogen atom.

In another embodiment, the chemiluminescent compound has the formula:

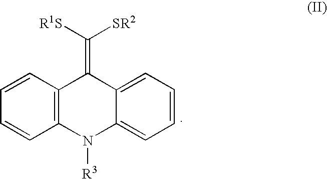

In some embodiments, R¹, R², R³, R⁴, R⁵, R⁶, R⁷, R⁸, R⁹, R¹⁰, and R¹¹ are hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, a linking moiety (-L-), or a substituent comprising a linking moiety (-L-). At least one of R¹ to R¹¹ includes a linking moiety or is a linking moiety (-L-). The linking moiety (-L-) is a bond, the reaction product of two reactive groups, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene. In some embodiment, R¹ and R² are not hydrogen. In other embodiments, R¹ or R² are -L- or comprise -L-. The groups R¹, R² and R³ are as defined above, in the compounds of Formula (I). In some embodiments, the compound of Formulas (I) or (II) have a linking moiety as a substituent on the R¹ or R² group. In some embodiments, the chemiluminescent moiety is an acridan kentenedithioacetal.

The linking moiety connects the chemiluminescent moiety to the solid support. The linking moiety may be attached to the solid support through a covalent bond. The covalent bond may be formed by contacting a reactive group on a linking moiety precursor with a reactive group on a solid support precursor. The solid support precursor may include a spacer moiety with a reactive group in order to increase chemical accessibility to the linking moiety precursor reactive group. By reacting the reactive groups of the linking moiety precursor and the solid support precursor, the chemiluminescent moiety is connected to the solid support. Exemplary classes of reactions are those proceeding under relatively mild conditions. These include, but are not limited to nucleophilic substitutions (e.g., reactions of amines and alcohols with acyl halides, active esters), electrophilic substitutions (e.g., enamine reactions), and additions to carbon-carbon and carbon-heteroatom multiple bonds (e.g., Michael reaction, Diels-Alder addition). These and other useful reactions are discussed in, for example, March, Advanced Organic Chemistry, 3rd Ed., John Wiley & Sons, New York, 1985; Hermanson, Bioconjugate Techniques, Academic Press, San Diego, 1996; and Feeney et al., Modification of Proteins; Advances in Chemistry Series, Vol. 198, American Chemical Society, Washington, D.C., 1982.

Useful reactive groups include, for example: (a) carboxyl groups and derivatives thereof including, but not limited to activated esters, e.g., N-hydroxysuccinimide esters, N-hydroxybenztriazole esters, acid halides, acyl imidazoles, thioesters, p-nitrophenyl esters, alkyl, alkenyl, alkynyl and aromatic esters, activating groups used in peptide synthesis and acid halides; (b) hydroxyl groups, which can be converted to esters, sulfonates, phosphoramidites, ethers, aldehydes, etc.; (c) haloalkyl groups, wherein the halide can be displaced with a nucleophilic group such as, for example, an amine, a carboxylate anion, thiol anion, carbanion, or an alkoxide ion, thereby resulting in the covalent attachment of a new group at the site of the halogen atom; (d) dienophile groups, which are capable of participating in Diels-Alder reactions such as, for example, maleimido groups; (e) aldehyde or ketone groups, allowing derivatization via formation of carbonyl derivatives, e g, imines, hydrazones, semicarbazones or oximes, or via such mechanisms as Grignard addition or alkyllithium addition; (f) sulfonyl halide groups for reaction with amines, for example, to form sulfonamides; (g) thiol groups, which can be converted to disulfides or reacted with acyl halides, for example; (h) amine or sulfhydryl groups, which can be, for example, acylated, alkylated or oxidized; (i) alkenes, which can undergo, for example, cycloadditions, acylation, Michael addition, etc; (j) epoxides, which can react with, for example, amines and hydroxyl compounds; and (k) phosphoramidites and other standard functional groups useful in nucleic acid synthesis. In an exemplary embodiment, the solid support precursor includes a reactive amine and the linking moiety precursor includes a reactive carboxyl group. The solid support precursor is then covalently bonded to the linking moiety precursor using any appropriate amide bond forming agent, such as those used in the art of peptide synthesis.

The reactive groups can be chosen such that they do not participate in, or interfere with, the reactions necessary to assemble or utilize the chemiluminescent moiety. Alternatively, a reactive group can be protected from participating in the reaction by the presence of a protecting group. Those of skill in the art understand how to protect a particular functional group such that it does not interfere with a chosen set of reaction conditions. For examples of useful protecting groups, see, for example, Greene et al., Protective Groups in Organic Synthesis, John Wiley & Sons, New York, 1991.

In some cases attachment will not involve covalent bond formation, but rather physical forces in which case the linking group remains unaltered. Physical forces imply attractive forces such as hydrogen bonding, electrostatic or ionic attraction, hydrophobic attraction such as base stacking, and specific affinity interactions such as biotin-streptavidin, antigen-antibody and nucleotide-nucleotide interactions.

In addition to the reactive group the linking moiety precursor and or solid support precursor may further include a spacer. In some embodiments, the spacer is on one or both sides of the bond formed by the reaction of the reactive group. The spacer may be a substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene. In another related embodiment, the spacer is selected from C₁-C₁₀ substituted or unsubstituted alkylene, 2 to 10 membered substituted or unsubstituted heteroalkylene, C₃-C₈ substituted or unsubstituted cycloalkylene, and 3 to 8 membered substituted or unsubstituted heterocycloalkylene. The spacer can be further defined as a bond, an atom, divalent groups and polyvalent groups, a straight or branched chain of atoms some of which can be part of a ring structure. The straight or branched chain can be substituted or unsubstituted and can be an alkylene or a heteroalkylene. The substituent usually contains from 1 to about 50 non-hydrogen atoms, more usually from 1 to about 30 non-hydrogen atoms. Examples for atoms included in the chain are selected from, but not limited to C, O, N, S, P, Si, B, and Se atoms. In another embodiment atoms comprising the chain are selected from C, O, N, P and S atoms. The number of atoms other than carbon in the chain is normally from 0-10. Halogen atoms can be present as substituents on the chain or ring.

In some embodiments, a linking moiety may conjugate a competition analyte to an analyte binder, which can be a first or a second analyte binder, or to a solid support. Such linking moiety is referred to herein as a bifunctional linker. The bifunctional linker includes reactive groups at the point of attachment contacting the competition analyte and reactive groups at the point of attachment contacting the solid support or the analyte binder. The reactive groups at either point of attachment of the bifunctional linker may be separated by a spacer as previously described. The reactive groups at both points of attachment of the bifunctional linker may react with the corresponding reactive groups of the competition analyte and the solid support or the competition analyte and the analyte binder. Thus, the competition analyte is conjugated to the solid support or the analyte binder. Any of the linking moieties and reactive groups previously described may be used to conjugate the competition analyte to the analyte binder or the solid support.

Kit embodiments provide a convenient means for supplying necessary reagents of the invention, ancillary reagents, apparatuses, instructions and/or other components necessary to implement the invention. In one aspect, a kit for detecting an analyte in a sample is provided.

The kit includes a first analyte binder conjugate that is conjugated to a peroxidase enzyme, and a solid support conjugate that is conjugated to a second analyte binder, a peroxide generating enzyme, and a chemiluminescent compound. In some embodiments, the kit includes a solution containing the peroxide generating enzyme substrate. The kit may include a peroxidase enhancer compound. In some embodiments, the peroxidase enhancer compound is p-phenylphenol, p-iodophenol, p-bromophenol, p-hydroxycinnamic acid, p-imidazolylphenol, acetaminophen, 2,3,-dichlorophenol, 2-naphthol, or 6-bromo-2-naphthol. In one embodiment, the chemiluminescent compound in the kit includes a chemiluminescent acridan moiety. In some embodiments, the chemiluminescent acridan moiety is a acridan ketenedithioacetal. The first analyte binder and the second analyte binder included in the kit may be antibodies. The kit may include a solution containing a peroxide decomposition agent. In some embodiments, the peroxide decomposition agent of the kit is a catalase.

Other materials useful in the performance of the assays can also be included in the kit, including test tubes, transfer pipettes, and the like. The kit may also include written instructions for the use of one or more of the reagents described herein. The invention contemplates additional kits packaged to deliver, instruct and otherwise aid the practitioner in the use of the invention. These additional kits include those for the use of diagnostic embodiments of the invention, and their construction is well known by those of skill in the art provided with the reagents set forth herein.

### Detection

Light emitted by the present method can be detected by any suitable known means such as a luminometer, x-ray film, high speed photographic film, a CCD camera, a scintillation counter, a chemical actinometer or visually. Each detection means has a different spectral sensitivity. The human eye is optimally sensitive to green light, CCD cameras display maximum sensitivity to red light, X-ray films with maximum response to either UV to blue light or green light are available. Choice of the detection device will be governed by the application and considerations of cost, convenience, and whether creation of a permanent record is required. In those embodiments where the time course of light emission is rapid, it is advantageous to perform the triggering reaction to produce the chemiluminescence in the presence of the detection device. As an example the detection reaction may be performed in a test tube or microwell plate housed in a luminometer or placed in front of a CCD camera in a housing adapted to receive test tubes or microwell plates.

In some embodiments, light is measured in an instrument for performing assays. Such an instrument comprises one or more reaction vessels for performing assays. The reaction vessels may comprise disposable wells, tubes or cartridges into which are dispensed samples and other reagents needed for performing tests. The instrument may further comprise pumps and injectors for dispensing liquids and particles. The instrument may further comprise means for transporting reaction vessels to one or more zones within the instrument. The instrument further comprises a light measurement device, typically a photomultiplier, as well as means for recording one or more characteristics of the light produced by a sample in an assay. The instrument may further comprise a data collection, analysis and storage system, typically a computer. Characteristics of the light that may be measured in an assay include peak intensity, integrated intensity for some or all of the light emitting period, rate of change of light intensity, spectral distribution, ratio of intensity at more than one wavelength, time to achieve peak intensity, or time to achieve some fraction of peak intensity.

### Uses

The present assay methods find applicability in many types of specific binding pair assays. Foremost among these are chemiluminescent enzyme linked immunoassays, such as an ELISA. Various assay formats and the protocols for performing the immunochemical steps are well known in the art and include both competitive assays and sandwich assays. Types of substances that can be assayed by immunoassay according to the present methods include proteins, peptides, antibodies, haptens, drugs, steroids and other substances that are generally known in the art of immunoassay.

The methods provided herein are also useful for the detection of nucleic acids. The presented methods may use enzyme-labeled nucleic acid probes. Exemplary methods include solution hybridization assays, DNA detection in Southern blotting, RNA by Northern blotting, DNA sequencing, DNA fingerprinting, colony hybridizations and plaque lifts, the conduct of which is well known to those of skill in the art.

In addition to the aforementioned antigen-antibody, hapten-antibody or antibody-antibody pairs, specific binding pairs also can include complementary oligonucleotides or polynucleotides, avidin-biotin, streptavidin-biotin, hormone-receptor, lectin-carbohydrate, IgG protein A, binding protein-receptor, nucleic acid-nucleic acid binding protein and nucleic acid-anti-nucleic acid antibody. Receptor assays used in screening drug candidates are another area of use for the present methods.

### III. Examples

The following example demonstrates an immunoassay of an analyte, Prostate Specific Antigen (PSA), wherein hydrogen peroxide is generated through the reaction of glucose oxidase with glucose, where the glucose oxidase is bound with the surface of the solid phase.

The antibodies used for this example were those found in the Hybritech® PSA Assay (Item No. 37200) of the Access® Immunoassay System (Beckman Coulter, Inc., Fullerton, Calif., USA). The antibodies in the described embodiment are used in the same orientation, that is, the Hybritech® solid phase capture antibody is located on the solid phase support surface and the Hybritech® conjugate antibody is used for the peroxidase conjugate. It should be noted that while this experiment utilized the Hybritech® PSA antibodies one skilled in the art will recognize that other suitable antibody pairs could be substituted so long as such antibody pair provided the ability to form a specific binding pair sandwich with the analyte antigen. Unique buffers are described. Buffers not described are obvious to one skilled in the art.

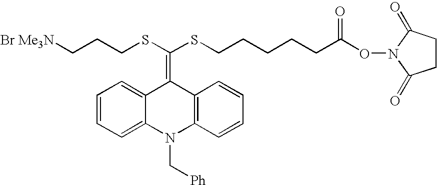

To prepare the base microparticles Bovine Serum Albumin (BSA) was biotinylated with 4× molar excess of biotin-LC-sulfoNHS (Pierce Biotechnology Inc., Rockford, Ill., USA). Unbound reactants were removed via desalting or dialysis. The biotin-BSA was then reacted with a 5× molar excess of Compound 17 in 20 mM sodium phosphate pH 7.2: DMSO 75:25, v/v) followed by desalting in the same buffer. The dual labeled (biotin and 17) BSA was then coupled with tosyl activated M280 microparticles (Invitrogen Corporation, Carlsbad, Calif., USA) in a 0.1M borate buffer pH 9.5 at a concentration of ca. 20 μg labeled BSA per mg of microparticles for 16-24 h at 40° C. After coupling the microparticles were stripped for 1 h at 40° C. with 0.2 M TRIS base, 2% SDS, pH ˜11. The stripping process was repeated one additional time. Microparticles were then suspended in a 0.1% BSA/TRIS buffered saline (BSA/TBS) buffer and streptavidin (SA) was added at approximately 15 μg SA per mg microparticles. Streptavidin was mixed with the microparticles for 45-50 min at room temperature. The microparticles were then washed three times and suspended in the same BSA/TBS. This describes the preparation of the base microparticles. Internal studies have shown these base microparticles are capable of binding approximately 5 μg of biotinylated capture antibody per mg of microparticles.

To prepare the actual test microparticles glucose oxidase (GOX), obtained from Sigma Aldrich, St. Louis, Mo., USA was biotinylated with a 5× molar excess of biotin-PEO₄—NHS, obtained from Pierce Biotechnology Inc., Rockford, Ill., USA. Unbound reactants were removed by desalting or dialysis. The PSA capture antibody was also biotinylated with a 5× molar excess of biotin-LC-sulfoNHS, (Pierce) and unbound reactants were removed by desalting or dialysis. To the base microparticles (above) the biotin capture antibody was added at 4 μg per mg of microparticles and the biotin GOX was added at 1 μg per mg of microparticles. The biotinylated proteins were mixed with the microparticles overnight at room temperature. After incubating all unbound reactants were removed by three washes in BSA/TBS.

The second analyte-specific binding partner, or antibody was prepared by first the activation of the Hybritech antibody with a 50× molar excess of DL-N-Acetylhomocysteine thiolactone (AHTL; Sigma-Aldrich) in 0.1M carbonate pH 9 for 1 h at room temperature. Excess reactant was removed by desalting into PBS plus 1 mM EDTA. At the same time HRP, (Roche Diagnostics, Indianapolis, Ind., USA) was activated with a 10× molar excess of sulfo-SMCC, (Pierce) for 1 h at room temperature. Excess reactant was removed by desalting into PBS. The activated Hybritech antibody and HRP were mixed together in a 1:5 (Ab:HRP) molar ratio and are allowed to react at room temperature for 1-2 hours. The reaction was stopped by the addition of a slight molar excess of 2-mercaptoethanol, then N-ethyl maleimide. The antibody-HRP second analyte-specific binding partner was then separated from unbound reactants by SEC. Trigger solution consisted of 0.1M sodium phosphate pH ˜7.2, 0.2M glucose, and 8 mM p-hydroxycinnamic acid.

To perform an assay the following stocks were prepared from the above described components. Microparticles were diluted to 1.75 mg/mL in BSA/TBS. Conjugate was diluted to 2 μg/mL in BSA/TBS. The reaction mixture was prepared by mixing the microparticle stock, a conjugate stock, buffer, and sample in the following ratio: 25:45:15:15 (volumes ratio of Microparticles:BSA/TBS:Conjugate:Sample) in this written order. The reaction mixture was incubated for 30 min at 37° C., then 100 μL of trigger solution was added and the light intensity recorded. To evaluate the effect of catalase (Sigma-Aldrich) the enzyme was added to the BSA/TBS added to the reaction at a concentration of 2 μg/ml.

Signal (light) was captured and quantified immediately after addition of the trigger solution with a PMT. The signal expressed as relative luminometer (light) units RLUs is provided in the following table.

| | |
|---|---|
| PSA | Average RLU |

| (ng/mL) | −catalase | +catalase |
|---|---|---|
| 0 | 1011 | 491 |
| 0.5 | 1184 | 1860 |
| 2 | 2054 | 712 |
| 10 | 3214 | 2532 |
| 75 | 15887 | 9792 |
| 150 | 21935 | 18878 |

What is claimed is:

1. A method of detecting an analyte in a sample comprising:
(a) contacting said analyte with a solid support conjugate and a first analyte binder conjugate, wherein said first analyte binder conjugate comprises a peroxidase enzyme conjugated to a first analyte binder, and said solid support conjugate comprises a solid support conjugated to a second analyte binder, a peroxide generating enzyme, and a chemiluminescent compound,
(b) allowing said analyte to bind to said first analyte binder and said second analyte binder thereby forming a detectable solid support bound analyte complex;
(c) contacting said detectable solid support bound analyte complex with a peroxide generating enzyme substrate thereby producing a peroxide;
(d) allowing said peroxidase enzyme to react with said peroxide thereby activating said chemiluminescent compound and producing a chemiluminescent signal; and
(e) detecting said chemiluminescent signal thereby detecting said analyte.

2. The method of claim 1, wherein said peroxidase enzyme is a horseradish peroxidase enzyme.

3. The method of claim 1, wherein
said peroxide generating enzyme is glucose oxidase, glycollate oxidase, hexose oxidase, cholesterol oxidase, aryl-alcohol oxidase, L-gulonolactone oxidase, galactose oxidase, pyranose oxidase, L-sorbose oxidase, pyridoxine oxidase, alcohol oxidase, L-2-hydroxy-acid oxidase, ecdysome oxidase, choline oxidase, aldehyde oxidase, xanthine oxidase, pyruvate oxidase, oxalate oxidase, glyoxylate oxidase, pyruvate oxidase, D-aspartate oxidase, L-aminoacid oxidase, amine oxidase, pyridoxaminephosphate oxidase, D-glutamate oxidase, ethanolamine oxidase, tyramine oxidase, putrascine oxidase, sarcosine oxidase, N-methylaminoacid oxidase, N-methyl-lysine oxidase, hydroxylnicotine oxidase, nitroethane oxidase, acetyl-indoxyl oxidase, urate oxidase, hydroxylamine oxidase, or sulphite oxidase; and
said peroxide generating enzyme substrate is glucose, glycollate, hexose, cholesterol, aryl-alcohol, L-gulonolactone, galactose, pyranose, L-sorbose, pyridoxine, alcohol, L-2-hydroxy-acid, ecdysome, choline, aldehyde, xanthine, pyruvate, oxalate, glyoxylate, pyruvate, D-aspartate, L-aminoacid, amine, pyridoxaminephosphate, D-glutamate, ethanolamine, tyramine, putrascine, sarcosine, N-methylaminoacid, N-methyl-lysine, hydroxylnicotine, nitroethane, acetyl-indoxyl, urate, hydroxylamine, or sulphite, respectively.

4. The method of claim 1, wherein said peroxide generating enzyme is glucose oxidase and said peroxide generating enzyme substrate is glucose.

5. The method of claim 1, further comprising contacting said detectable solid support bound analyte complex with a peroxidase enhancer compound in step (c).

6. The method of claim 5, wherein the peroxidase enhancer compound is p-phenylphenol, p-iodophenol, p-bromophenol, p-hydroxycinnamic acid, p-imidazolylphenol, acetaminophen, 2,3,-dichlorophenol, 2-naphthol, or 6-bromo-2-naphthol.

7. The method of claim 1, wherein the first analyte binder and the second analyte binder are binding proteins.

8. The method of claim 1, wherein the first analyte binder and the second analyte binder are antibodies.

9. The method of claim 1, wherein the chemiluminescent compound comprises a chemiluminescent acridan moiety.

10. The method of claim 1, further comprising contacting said detectable solid support bound analyte complex with a peroxide decomposition agent.

11. The method of claim 10, wherein said peroxide decomposition agent is an enzyme.

12. The method of claim 10, wherein said peroxide decomposition agent is a catalase.

13. The method of claim 12, wherein said catalase is present with said detectable solid support bound analyte complex at a concentration of 2 μg/ml.

14. The method of claim 10, wherein said contacting said detectable solid support bound analyte complex with said peroxide decomposition agent is performed prior to step (c).

15. A method of detecting an analyte in a sample comprising:
(a) contacting said analyte with a solid support conjugate and a first analyte binder conjugate, wherein said first analyte binder conjugate comprises a peroxidase enzyme conjugated to a first analyte binder and said first analyte binder is bound to a competition analyte, and said solid support conjugate comprises a solid support conjugated to a second analyte binder, a peroxide generating enzyme, and a chemiluminescent compound,
(b) allowing said analyte and said first analyte binder conjugate to bind competitively to said second analyte binder, wherein the binding of said first analyte binder conjugate to said second analyte binder forms a detectable solid support bound analyte complex;
(c) contacting said detectable solid support bound analyte complex with a peroxide generating enzyme substrate thereby producing a peroxide;
(d) allowing said peroxidase enzyme to react with said peroxide thereby activating said chemiluminescent compound and producing a chemiluminescent signal; and
(e) detecting said chemiluminescent signal thereby detecting said analyte.

16. A solid support conjugate comprising a solid support conjugated to a chemiluminescent compound, a hydrogen peroxide generating enzyme, and an analyte binder.

17. The solid support conjugate of claim 16, wherein said hydrogen peroxide generating enzyme is a glucose oxidase.

18. The solid support conjugate of claim 16, wherein the chemiluminescent compound comprises a chemiluminescent acridan moiety.

19. The solid support conjugate of claim 16, wherein said chemiluminescent compound has the formula: 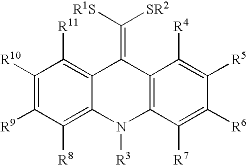
wherein
R¹ and R² are independently substituted or unsubstituted alkyl, substituted or unsubstituted alkenyl, substituted or unsubstituted alkynyl, substituted or unsubstituted aryl, or substituted or unsubstituted aralkyl;
R³ is substituted or unsubstituted alkyl, substituted or unsubstituted alkenyl, substituted or unsubstituted alkynyl, substituted or unsubstituted aryl, substituted or unsubstituted aralkyl, alkoxyalkyl, carboxyalkyl or alkylsulfonic acid, wherein R³ is optionally joined with R⁷ or R⁸ to form a 5 or 6-membered ring
R⁴, R⁵, R⁶, R⁷, R⁸, R⁹, R¹⁰, and R¹¹ are independently hydrogen, substituted or unsubstituted alkyl, substituted or unsubstituted aryl, aralkyl, alkenyl, alkynyl, alkoxy, aryloxy, halogen, amino, substituted amino, substituted or unsubstituted carboalkoxy, carboxamide, cyano, or sulfonate, wherein pairs of adjacent groups of R⁴, R⁵, R⁶, R⁷, R⁸, R⁹, R¹⁰, and R¹¹ are optionally joined to form a carbocyclic or heterocyclic ring system;
and wherein at least one of the groups of R¹ to R¹¹ comprises a linking moiety that links the compound to said solid support.

20. The solid support conjugate of claim 16, wherein said analyte binder is an antibody.

21. A kit for detecting an analyte in a sample comprising:
a. a first analyte binder conjugate comprising a first analyte binder conjugated to a peroxidase enzyme, and
b. a solid support conjugate comprising a solid support conjugated to a second analyte binder, a peroxide generating enzyme, and a chemiluminescent compound.

22. The kit of claim 21, further comprising a solution comprising a peroxide generating enzyme substrate.

23. The kit of claim 22, wherein said solution further comprises a peroxidase enhancer compound.

24. The kit of claim 23, wherein the peroxidase enhancer compound is p-phenylphenol, p-iodophenol, p-bromophenol, p-hydroxycinnamic acid, p-imidazolylphenol, acetaminophen, 2,3,-dichlorophenol, 2-naphthol, or 6-bromo-2-naphthol.

25. The kit of claim 21, wherein the chemiluminescent compound comprises a chemiluminescent acridan moiety.

26. The kit of claim 21, wherein the first analyte binder and the second analyte binder are antibodies.

27. The kit of claim 21, further comprising a solution comprising a peroxide decomposition agent.

28. The kit of claim 27, wherein said peroxide decomposition agent is a catalase.

29. A method of detecting an analyte in a sample comprising:
(a) contacting said sample with a solid support conjugate and an analyte-peroxidase conjugate, wherein said analyte-peroxidase conjugate comprises a peroxidase enzyme conjugated to the analyte or a homolog of the analyte and said solid support conjugate comprises a solid support conjugated to a first analyte binder, a peroxide generating enzyme, and a chemiluminescent compound,
(b) allowing said analyte in said sample and said analyte-peroxidase conjugate to bind competitively to said first analyte binder, wherein the binding of said first analyte binder conjugate to said analyte-peroxidase conjugate forms a detectable solid support bound analyte complex;
(c) contacting said detectable solid support bound analyte complex with a peroxide generating enzyme substrate thereby producing a peroxide;
(d) allowing said peroxidase enzyme to react with said peroxide thereby activating said chemiluminescent compound and producing a chemiluminescent signal; and
(e) detecting said chemiluminescent signal thereby detecting said analyte.
